(19) United States
(12) Patent Application Publication
(10) Pub. No.: US 2025/0031577 A1
(43) Pub. Date: Jan. 23, 2025

(54) INTEGRATED ULTRASONIC TRANSDUCER MICROELECTRONIC ASSEMBLY METHOD, ULTRASOUND PROBE, AND SYSTEM

(71) Applicant: GE Precision Healthcare LLC, Wauwatosa, WI (US)

(72) Inventor: Kieran Wall, Niskayuna, NY (US)

(21) Appl. No.: 18/354,486

(22) Filed: Jul. 18, 2023

(51) Int. Cl. H10N 30/06; A61B 8/00; B06B 1/06; H10N 30/088; H10N 30/87

(52) U.S. Cl. CPC H10N 30/06; A61B 8/4227; A61B 8/4236; A61B 8/4455; A61B 8/4472; B06B 1/0622; H10N 30/088; H10N 30/875; B06B 2201/76

(57) ABSTRACT

A method of manufacturing an ultrasonic transducer microelectronic assembly comprises, forming a transducer directly on a flex circuit of a main circuit assembly without anything in between, wherein the transducer is a piezoelectric transducer. In some examples, the method further includes directly integrating an electrode pattern into the flex circuit. In some examples, the method includes attaching a piezoelectric crystal body directly to the electrode pattern using one of thermocompression, ACF, ACP, and non-conductive epoxy.

### FIELD

Embodiments of the subject matter disclosed herein relate to a method of manufacturing an ultrasound transducer assembly for an ultrasound probe and a system for use thereof.

### BACKGROUND

An ultrasound probe may include various components mechanically, conductively, or chemically coupled to form an ultrasound transducer within a housing. For example, the ultrasound transducer structure may include a crystal/ceramic element with piezoelectric properties. Positive and ground electrode layers may be applied to the back and front faces, respectively, of the element. An acoustic backing material (also referred to as an acoustic absorber or a carrier) may be adhered to back of the element behind the positive electrode, and the ground electrode may be overlaid with one or more matching layers and/or a lens. The ultrasound transducer may be communicatively coupled to a main circuit board assembly of an ultrasound imaging system. In a typical ultrasound probe, the transducer is connected to the main circuit board assembly through a large, detachable connector. In this way, the ultrasound probe may receive and transmit ultrasonic acoustic waves to image a subject, such as a patient.

### BRIEF DESCRIPTION

In one embodiment, a method, comprising: forming a transducer directly on a flex circuit of a main circuit assembly without anything in between, wherein the transducer is a piezoelectric transducer.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present invention will be better understood from reading the following description of non-limiting embodiments, with reference to the attached drawings, wherein below:

FIG. 1 shows an ultrasound imaging system according to an exemplary embodiment.

FIG. 2 shows a first example of a flexible printed circuit board for a transducer assembly, according to an exemplary embodiment.

FIG. 3 shows an ultrasound transducer microelectronic assembly method, according to an exemplary embodiment.

FIG. 4 shows a first example of an ultrasound probe, according to an exemplary embodiment.

FIG. 5 shows a second example of a flexible printed circuit board for a transducer assembly, according to an exemplary embodiment.

FIG. 6 shows a second example of an ultrasound probe, according to an exemplary embodiment.

FIG. 7 shows a third example of a flexible printed circuit board for a transducer assembly, according to an exemplary embodiment.

FIG. 8 shows a third example of an ultrasound probe, according to an exemplary embodiment.

FIG. 9 is a flow chart illustrating a first method of manufacture for an ultrasound probe including an ultrasound transducer microelectronic assembly.

FIG. 10 is a flow chart illustrating a second method of manufacture for an ultrasound probe including an ultrasound transducer microelectronic assembly.

FIG. 11 is a flow chart illustrating a third method of manufacture for an ultrasound probe including an ultrasound transducer microelectronic assembly.

FIG. 12 is a fourth example of a flexible printed circuit board for a transducer assembly, according to an exemplary embodiment.

### DETAILED DESCRIPTION

The following description relates to various embodiments of an ultrasound transducer microelectronic assembly method, an ultrasound probe including the ultrasound transducer microelectronic assembly, and a medical imaging system including the ultrasound probe. The ultrasound transducer microelectronic assembly method allows for manufacture of an ultra-compact ultrasound probe, such as a patch probe. The method achieves a high degree of integration and compactness by avoiding use of one or more detachable connectors that in typical ultrasound probes conductively couple a main circuit board assembly with piezoelectric elements comprising the transducer. The commonly used connectors are bulky and prone to disconnection when dropped. Consequently, by avoiding the use of connectors, a sturdier and more robust design is realized at the same time enabling substantial packaging reduction.

The disclosed ultrasound transducer microelectronic assembly method includes forming a transducer directly on a flex circuit of a main circuit board assembly without anything in between, wherein the transducer is a piezoelectric transducer. The disclosure addresses current challenges related to manufacture of ultra-compact ultrasound probe designs by increasing durability and reducing overall probe dimensions. This is achieved by directly integrating an electrode pattern into the flex circuit. In one example, the electrode pattern may be an electrode of the transducer, such as, for example, a top electrode (also called an active electrode) of the transducer, In another example, the electrode pattern may be an electrode on two sides of the transducer, such as, for example, the top electrode and a bottom electrode (also called a ground electrode) of the transducer. In further example, the electrode pattern may be an electrode, such as, for example, the top electrode or the bottom electrode, and a ground return connection. By integrating the electrode pattern into the flex circuit, the transducer may be formed directly on the main circuit assembly, avoiding the use of special connectors, cables, wires, etc. For example, a piezoelectric crystal body may be attached directly to the electrode pattern using standard attachment techniques and additional layers may be applied thereto to form the transducer assembly. The method may include folding the flex circuit and transducer formed thereon so as to vertically stack the transducer and the main circuit board. A battery may be conductively coupled, interposed between the transducer assembly and the main circuit assembly, and the components packaged in a housing.

The main circuit board assembly may be designed with custom ASICs, including an acquisition system and a processing system, configured to acquire image data corresponding to a subject and to process the image data to generate one or more datasets corresponding to the subject. The ultrasound probe via the main circuit board assembly may integrate wirelessly with a user interface, such as a tablet or smart phone environment, and a medical records system, such as a cloud-based system. In this way, the disclosure provides for a compact, medical imaging system.

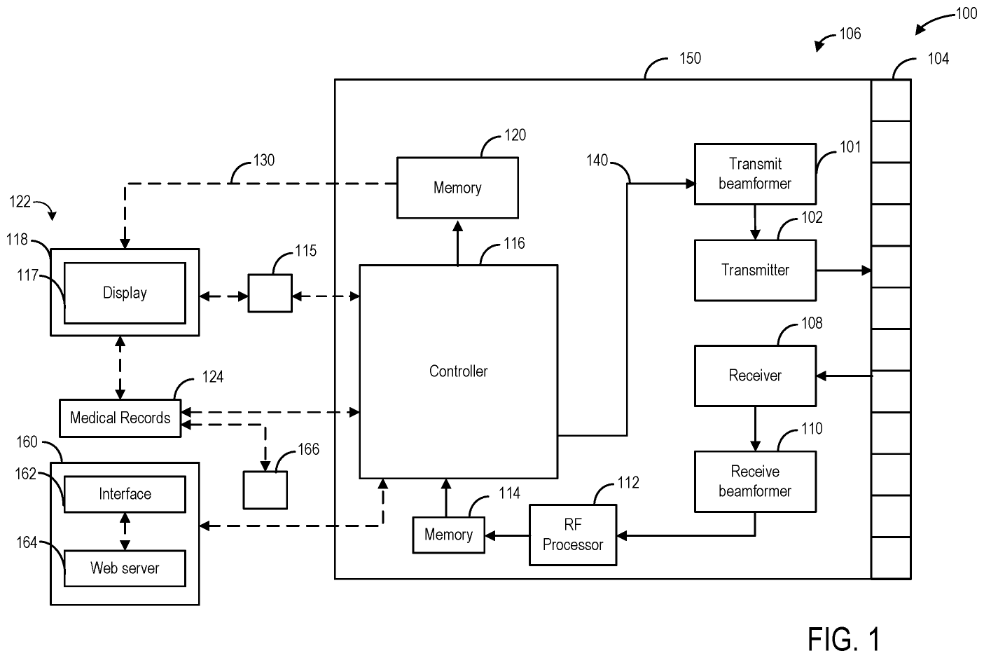

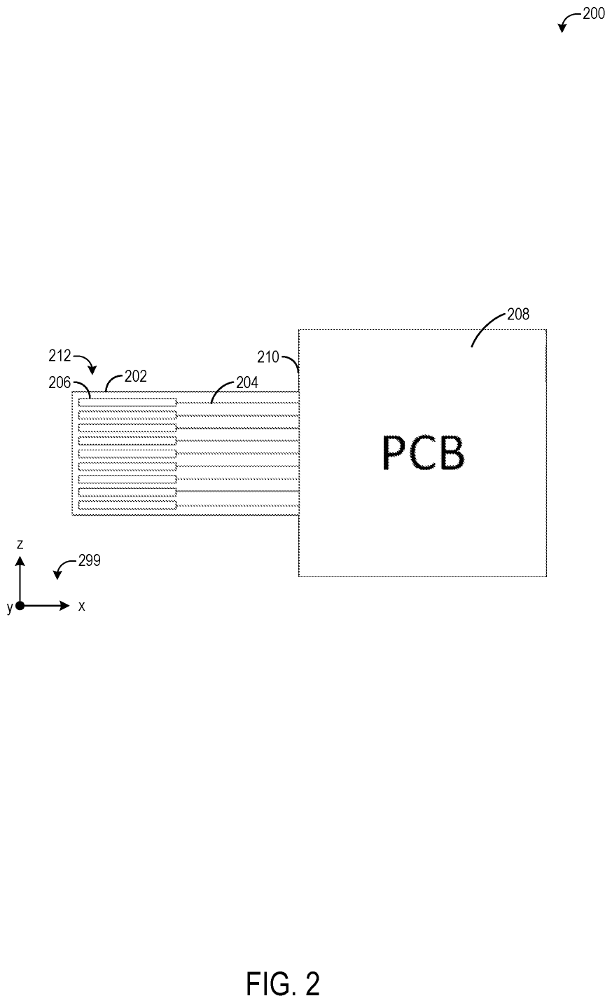

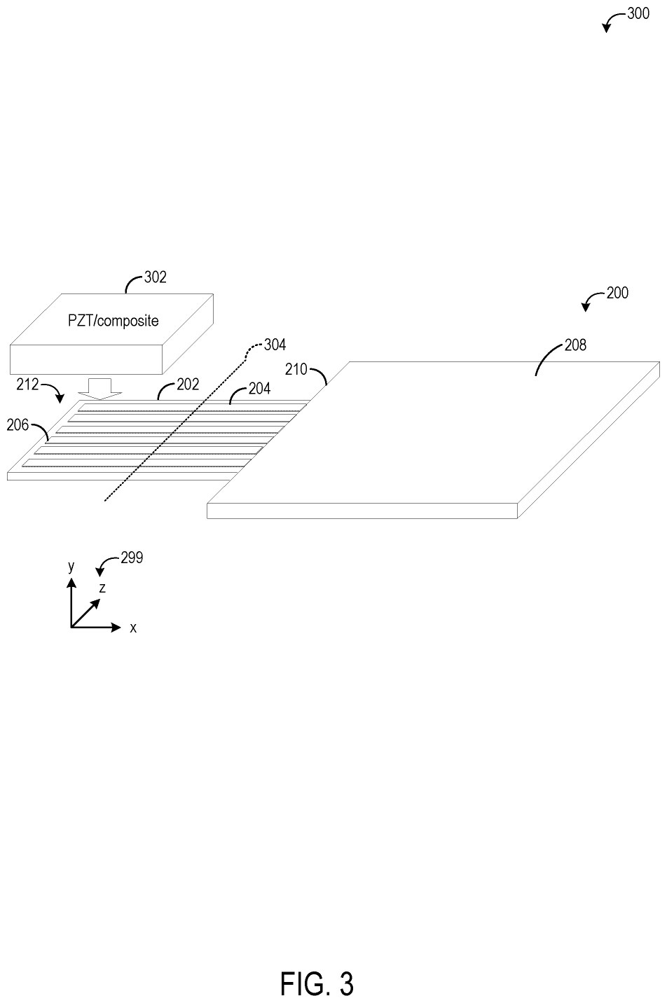

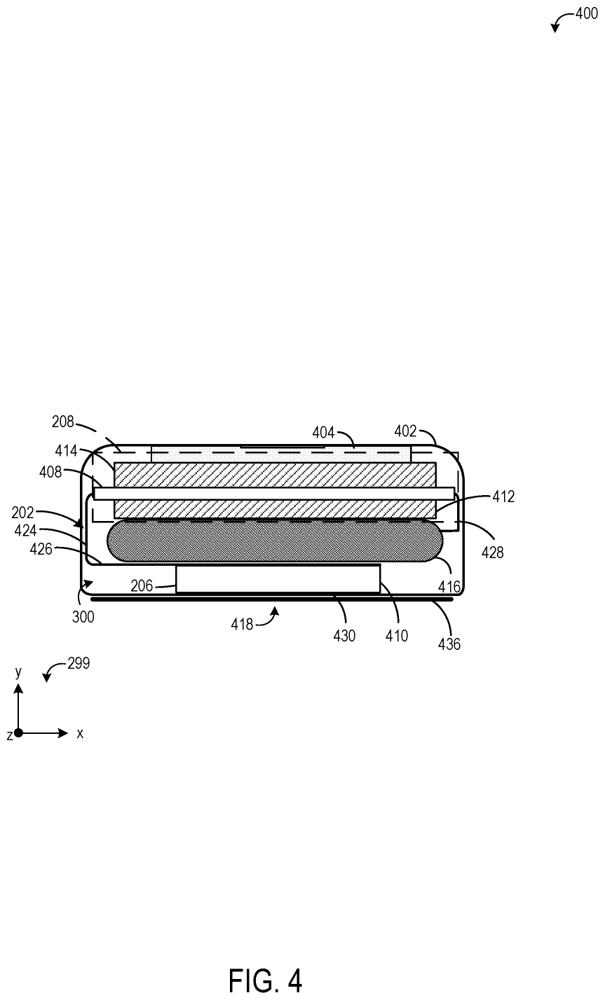

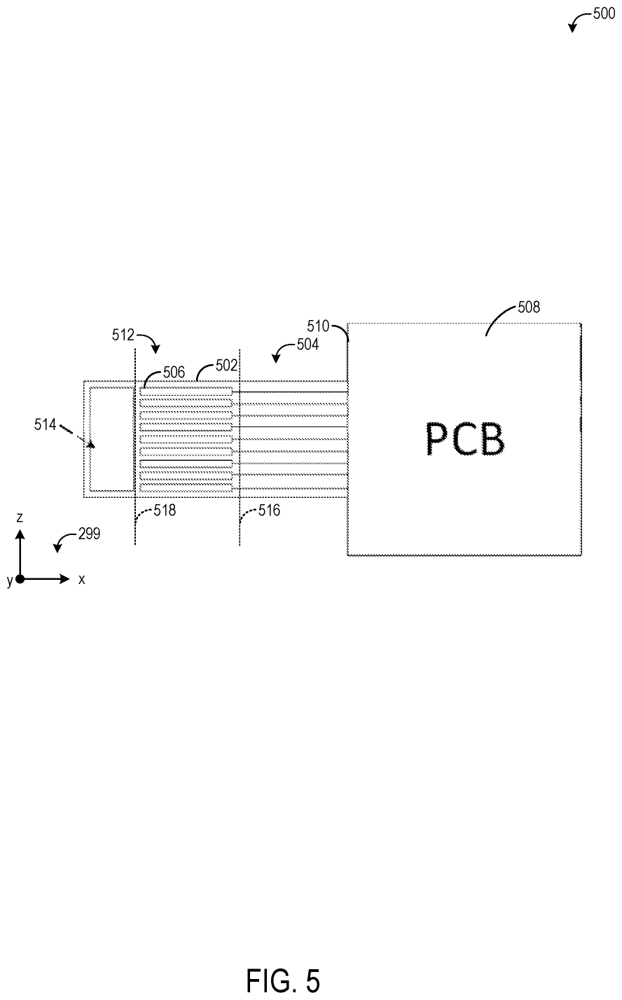

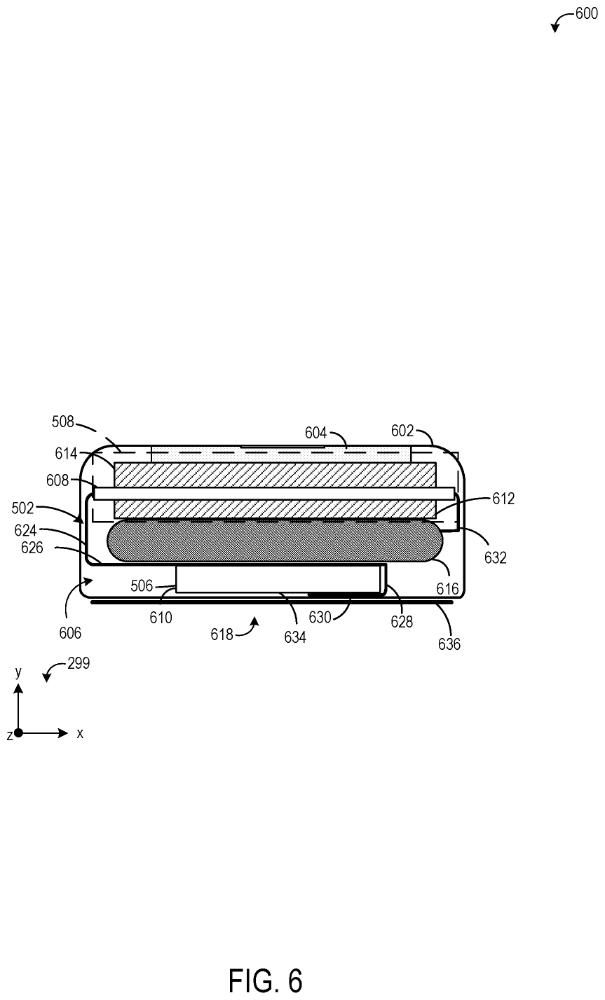

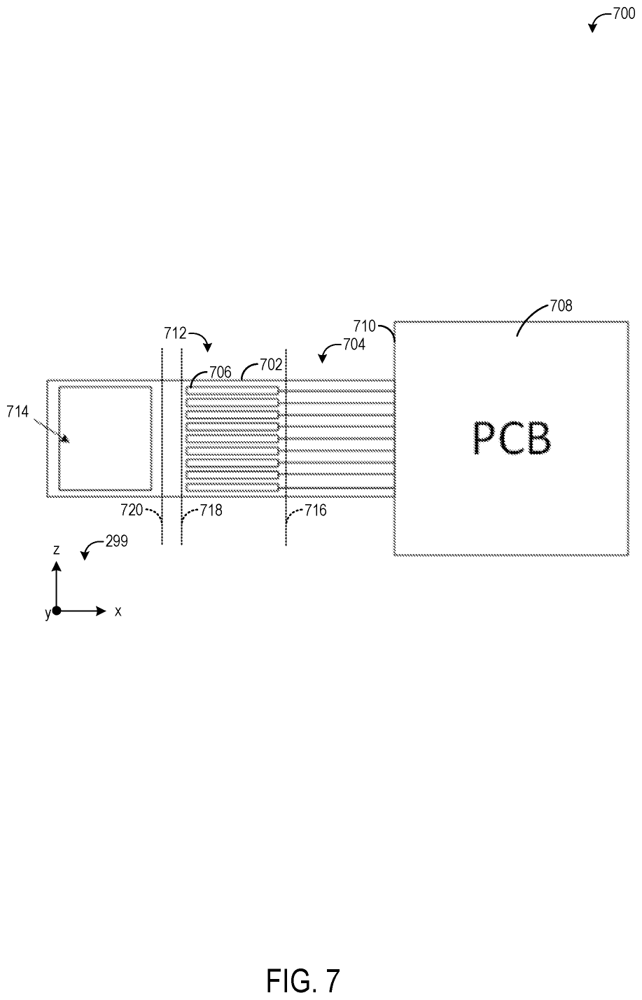

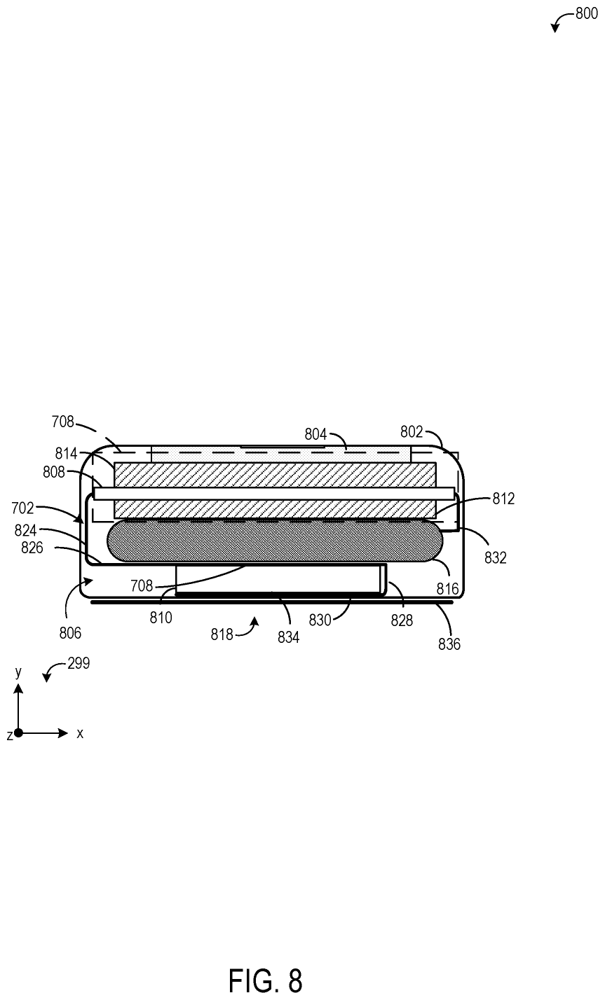

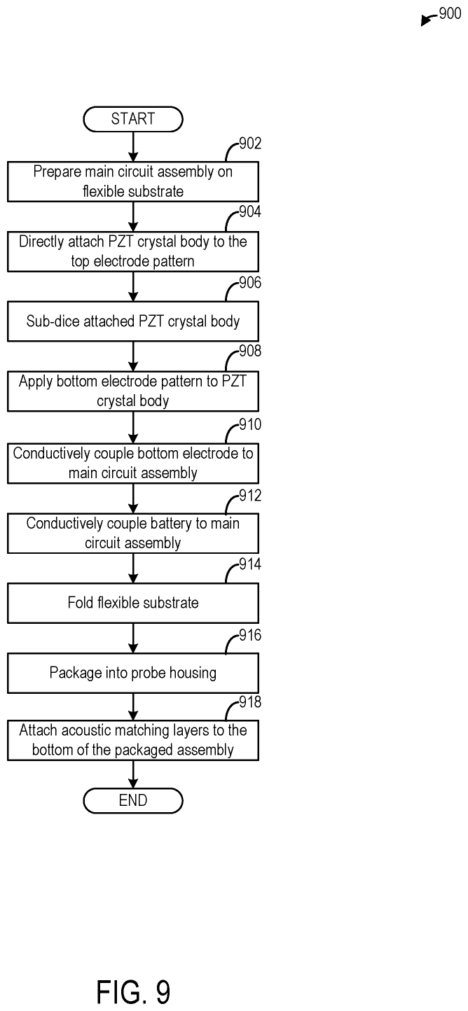

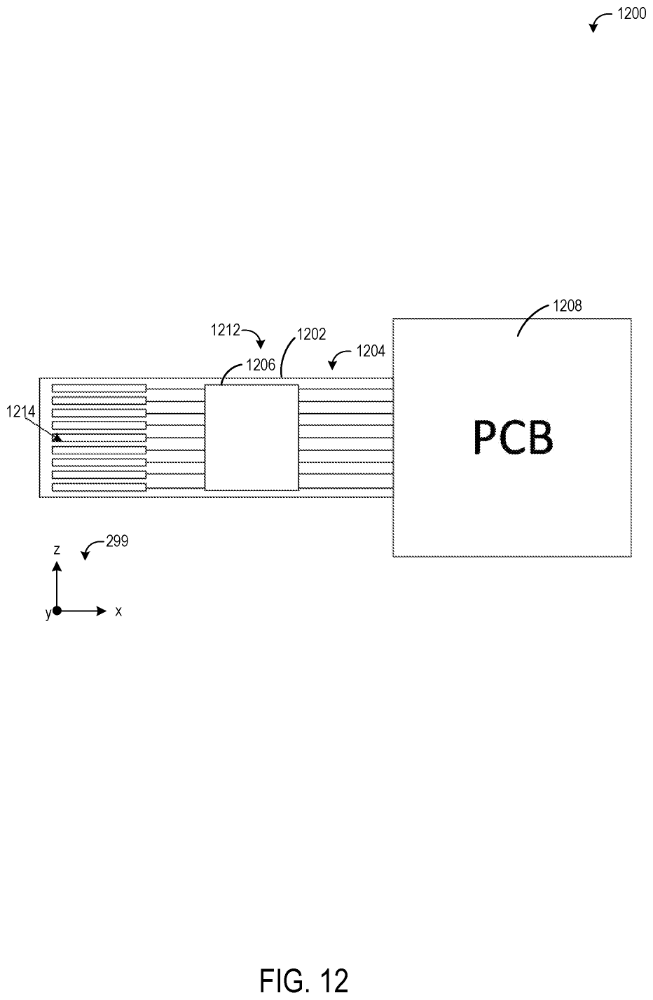

One example of an ultrasound imaging system including the ultrasound probe is depicted in FIG. 1. An example of a flexible printed circuit board that may be included in the disclosed ultrasound transducer microelectronic assembly is shown in FIG. 2. An example of the disclosed ultrasound transducer microelectronic assembly method is shown in FIG. 3. A first example of an ultrasound probe including the disclosed ultrasound transducer microelectronic assembly is shown in FIG. 4. A second example of a flexible printed circuit board that may be included in the disclosed ultrasound transducer microelectronic assembly is shown in FIG. 5. A second example of an ultrasound probe including the ultrasound transducer microelectronic assembly is shown in FIG. 6. A third example of a flexible printed circuit board that may be included in the disclosed ultrasound transducer microelectronic assembly is shown in FIG. 7. A third example of an ultrasound probe including the ultrasound transducer microelectronic assembly is shown in FIG. 8. Corresponding methods for manufacturing the ultrasound probe including the ultrasound transducer microelectronic assembly are depicted in FIGS. 9-11. A fourth example of a flexible printed circuit board that may be included in the disclosed ultrasound transducer microelectronic assembly is shown in FIG. 12.

FIG. 1 depicts a block diagram of a system 100 according to one embodiment. In the illustrated embodiment, the system 100 is an imaging system and, more specifically, an ultrasound imaging system. As shown, the system 100 includes an ultrasound probe 106, a user interface 122, and a medical records system 124, and a remote connectivity subsystem 160. A plurality of dashed lines 130 represent communicative couplings between components of system 100. A plurality of solid lines 140 represents communicative couplings between components of ultrasound probe 106. The components may be separate but located within a common room, or may be remotely located with respect to one another. For example, one or more of the modules described herein may operate in a data server that has a distinct and remote location with respect to other components of the system 100, such as a probe and user interface. In one example, the ultrasound probe 106 may be a patch probe.

In the illustrated embodiment, the ultrasound probe 106 comprises an ultrasound control system integrated into a flexible printed circuit board (PCB) 150 and a piezoelectric transducer comprising an array of elements 104, for example, piezoelectric elements including piezoceramics, high-dielectric ceramics, single crystals, etc. The PCB 150 may include a controller 116 that may be part of a single processing unit (e.g., processor) or distributed across multiple processing units. The controller 116 is configured to control operation of the system 100 including, for example, image acquisition and image processing. In one example, the controller 116 of the PCB 150 controls a transmit beamformer 101 and a transmitter 102 that drives the elements 104 within the ultrasound probe 106 to emit ultrasonic signals (e.g., continuous or pulsed) into a body or volume (not shown) of a subject. The controller 116 receives control signals from a receiver 108, a receive beamformer 110, a radio frequency (RF) processor 112, and a memory 114. The elements 104 and the ultrasound probe 106 may have a variety of geometries. The ultrasonic signals are back-scattered from structures in a body, for example, an inserted needle, to produce echoes that return to the elements 104. The echoes are received by the receiver 108. The received echoes are provided to the receive beamformer 110 that performs beamforming and outputs a radio frequency (RF) signal. The RF signal is then provided to the RF processor 112 that processes the RF signal. Alternatively, the RF processor 112 may include a complex demodulator (not shown) that demodulates the RF signal to form I/Q data pairs representative of the echo signals. The RF or I/Q signal data may then be provided directly to the memory 114 for storage (for example, temporary storage).

For example, the controller 116 may include an image-processing module that receives image data (e.g., ultrasound signals in the form of RF signal data or I/Q data pairs) and processes image data. For example, the image-processing module may process the ultrasound signals to generate two-dimensional (2D) slices or frames of ultrasound information (e.g., ultrasound images) or ultrasound waveforms (e.g., continuous or pulse wave Doppler spectrum or waveforms) for displaying to the operator. The image-processing module may be configured to perform one or more processing operations according to a plurality of selectable ultrasound modalities on the acquired ultrasound information. By way of example, the ultrasound modalities may include color-flow, acoustic radiation force imaging (ARFI), B-mode, A-mode, M-mode, spectral Doppler, acoustic streaming, tissue Doppler module, C-scan, and elastography. Further, in some examples, the one or more processing operations may include one or more image transforms, such as a Radon transform for identifying linear features in the ultrasound images.

Acquired ultrasound information may be processed in real-time during an imaging session (or scanning session) as the echo signals are received. Additionally or alternatively, the ultrasound information may be stored temporarily in the memory 114 during an imaging session and processed in less than real-time in a live or off-line operation. An image memory 120 is included for storing processed slices or waveforms of acquired ultrasound information that are not scheduled to be displayed immediately. The image memory 120 may comprise any known data storage medium, for example, a permanent storage medium, removable storage medium, and the like. Additionally, the image memory 120 may be a non-transitory storage medium.

In operation, an ultrasound system may acquire data, for example, 2D data sets, spectral Doppler data sets, and/or volumetric data sets by various techniques (for example, three-dimensional (3D) scanning, real-time 3D imaging, volume scanning, 2D scanning with probes having positioning sensors, freehand scanning using a voxel correlation technique, scanning using 2D or matrix array probes, and the like). Ultrasound spectrum (e.g., waveforms) and/or images may be generated from the acquired data (at the controller 116) and displayed to the operator or user on a display device 118.

The controller 116 is operably connected to the user interface 122 that enables an operator to control at least some of the operations of the system 100. The ultrasound probe 106 may be communicatively coupled to the user interface 122 via one or more wireless networks. The controller 116 may also be coupled to the remote connectivity subsystem 160 including a remote connectivity interface 162 and a web server 164. Similarly, the controller 116 may be communicatively coupled to the medical records system 124 configured to receive and/or store ultrasound image data. The medical records system 124 interacts with an imaging workstation 166. Thus, transmission of data and signals between the ultrasound probe 106, the user interface 122, the medical records system 124, and the remote connectivity subsystem for generating, interpreting, and managing ultrasound images may be enabled via the one or more wireless networks. The user interface 122 may include hardware, firmware, software, or a combination thereof that enables an individual (e.g., an operator) to directly or indirectly control operation of the system 100 and the various components thereof. As shown, the user interface 122 includes the display device 118 having a display area 117. In some embodiments, the user interface 122 may also include one or more user input devices 115, such as a physical keyboard, mouse, and/or touchpad. In one embodiment, a touchpad may be configured to the controller 116 and display area 117, such that when a user moves a finger/glove/stylus across the face of the touchpad, a cursor atop the ultrasound image or Doppler spectrum on the display device 118 moves in a corresponding manner.

In an exemplary embodiment, the display device 118 is a touch-sensitive display (e.g., touchscreen) that can detect a presence of a touch from the operator on the display area 117 and can also identify a location of the touch in the display area 117. The touch may be applied by, for example, at least one of an individual's hand, glove, stylus, or the like. As such, the touch-sensitive display may also be characterized as a user input device that is configured to receive inputs from the operator (such as a request to adjust or update an orientation of a displayed image). The display device 118 also communicates information from the controller 116 to the operator by displaying the information to the operator. The display device 118 and/or the user interface 122 may also communicate audibly. The display device 118 is configured to present information to the operator during or after the imaging or data acquiring session. The information presented may include ultrasound images (e.g., one or more 2D frames), graphical elements, measurement graphics of the displayed images, user-selectable elements, user settings, and other information (e.g., administrative information, personal information of the patient, and the like).

In addition to the image-processing module, the controller 116 may also include one or more of a graphics module, an initialization module, a tracking module, and an analysis module. The image-processing module, the graphics module, the initialization module, the tracking module, and/or the analysis module may coordinate with one another to present information to the operator during and/or after the imaging session. For example, the image-processing module may be configured to display an acquired image on the display device 118. and the graphics module may be configured to display designated graphics along with the displayed image, such as selectable icons (e.g., image rotation icons) and measurement parameters (e.g., data) relating to the image.

The screen of a display area 117 of the display device 118 is made up of a series of pixels which display the data acquired with the ultrasound probe 106. The acquired data includes one or more imaging parameters calculated for each pixel, or group of pixels (for example, a group of pixels assigned the same parameter value), of the display, where the one or more calculated image parameters includes one or more of an intensity, velocity (e.g., blood flow velocity), color flow velocity, texture, graininess, contractility, deformation, and rate of deformation value. The series of pixels then make up the displayed image and/or Doppler spectrum generated from the acquired ultrasound data.

FIGS. 2-8 show example configurations with relative positioning of the various components. If shown directly contacting each other, or directly coupled, then such elements may be referred to as directly contacting or directly coupled, respectively, at least in one example. Similarly, elements shown contiguous or adjacent to one another may be contiguous or adjacent to each other, respectively, at least in one example. As an example, components laying in face-sharing contact with each other may be referred to as in face-sharing contact. As another example, elements positioned apart from each other with only a space there-between and no other components may be referred to as such, in at least one example. As yet another example, elements shown above/below one another, at opposite sides to one another, or to the left/right of one another may be referred to as such, relative to one another. Further, as shown in the figures, a topmost element or point of element may be referred to as a “top” of the component and a bottommost element or point of the element may be referred to as a “bottom” of the component, in at least one example. As used herein, top/bottom, upper/lower, above/below, may be relative to a vertical axis of the figures and used to describe positioning of elements of the figures relative to one another. As such, elements shown above other elements are positioned vertically above the other elements, in one example. As yet another example, shapes of the elements depicted within the figures may be referred to as having those shapes (e.g., such as being circular, straight, planar, curved, rounded, chamfered, angled, or the like). Further, elements shown intersecting one another may be referred to as intersecting elements or intersecting one another, in at least one example. Further still, an element shown within another element or shown outside of another element may be referred as such, in one example.

An axis system 299, including y-, x-, and z-axes, is depicted in FIG. 2, and in following figures, for indicating relative positioning of components with respect to one another. In the examples, the y-axis may be a vertical axis (e.g., parallel to a gravitational axis), the x-axis may be a lateral axis (e.g., a horizontal axis), and the z-axis may be a longitudinal axis. However, the axes may have other orientations in other examples.

Referring now to FIG. 2, a flexible printed circuit board 200 that may be included in a transducer assembly is schematically depicted according to one embodiment. In one example, the flexible printed circuit board 200 may be the same or similar to the PCB 150 included in the ultrasound probe 106 shown in FIG. 1.

The flexible printed circuit board 200 may include a flex circuit 202 that is integral with a main circuit board assembly (PCB) 208. In one example, an electrode pattern 206 may be directly integrated into the flex circuit 202. In one example, the flex circuit 202 is an elongate flexible segment having a plurality of electrical traces 204 routed therethrough. The electrical traces 204 are conductively coupled to the electrode pattern 206. In one example, the electrode pattern 206 is a top electrode 212. In one example, the elongate flexible segment comprising the flex circuit 202 extends laterally from a side 210 of the PCB 208.

In some examples, the flex circuit 202 may be made of a thin layer of polymide or any other a type of plastic that may withstand high temperatures and is highly resistant to chemicals and radiation. In some examples, a circuit may then printed onto the polymide layer using copper traces or conductive ink, which form the electrical traces 204 and the top electrode 212. Additionally or alternatively, copper foil may be rolled and annealed to the polymide using an adhesive. In some examples, the copper may be coated with a photo-sensitive layer, which is then exposed and etched to give the desired pattern of conductors and termination pads.

In one example, the flexible printed circuit board 200 may be a flex-flex board such that the flex circuit 202 and the PCB 208 are a flexible construction. In other examples, the PCB 208 may be a rigid-flex board such that the flex circuit 202 is a flexible construction and the PCB 208 is a rigid construction. In some examples, the PCB 208 may comprise a single-sided board. In other examples, the PCB 208 may comprise a double-sided board. In yet other examples, the PCB 208 may comprise a multi-layered board.

In one example, the PCB 208 may comprise the main circuit board of the ultrasound control system described with reference to FIG. 1. For example, the PCB 208 may include one or more of transmitter and receiver channels for generating and processing ultrasound signals, analog-to-digital converters for converting the analog transducer signals into digital signals, digital circuitry for performing beamforming, filtering, and other algorithms for processing the ultrasound data, memory including RAM and/or flash memory, Complex Programmable Logic Devices (CPLD), and control circuity including one or more microcontrollers. The PCB 208 may be prepared with custom ASICs to integrate ultrasound control functions, increase performance, and increase efficiency.

Referring now to FIG. 3, a transducer assembly 300 is schematically depicted according to one embodiment. In particular, the transducer assembly 300 is shown including a method of manufacture. The transducer assembly 300 comprises the flexible printed circuit board 200 and a piezoelectric crystal body 302. Components of the flexible printed circuit board 200 introduced with reference to FIG. 2 are numbered the same and will not be reintroduced.

The piezoelectric crystal body 302 may be directly attached to the flex circuit 202. Specifically, the piezoelectric crystal body 302 is directly attached to the electrode pattern 206 comprising the top electrode 212. The piezoelectric crystal body 302 uses the flex circuit 202 and the electrode pattern 206 integrated therein as the transducer element conductor, without applying a conductive coating or electrode pattern to the piezoelectric material. The piezoelectric crystal body 302 may be attached to the flex circuit 202 using standard attachment strategies including, for example, thermal compression, anisotropic conductive paste (ACP), anisotropic conductive film (ACF), ultrasonic bonding, non-conductive epoxy, and so on. In this way, the piezoelectric crystal body 302 is directly attached to the main processing system, e.g. PCB 208, PCB 150, without anything in between, and without using a detachable connector, cables, or other standard conductive couplings.

The piezoelectric crystal body 302 is a piezoelectric material, such as lead zirconate titanate (PZT). In one example, the piezoelectric crystal body 302 may be a single crystal. In one example, the piezoelectric crystal body 302 may be a composite. An acoustic stack may be attached preprocessed, or alternately formed on the piezoelectric crystal body 302 during manufacture of the transducer assembly 300. Transducer manufacturing on the flex may in one incarnation include sub-dicing elements after crystal to flex attachment, either with or without a subsequent kerf filling step. In addition to the piezoelectric crystal body 302, the acoustic stack may include one or more other layers, such as, but not limited to, backing material, one or more matching layers, and lens materials. The specifications of the transducer assembly may be selected based on the application.

With the piezoelectric crystal body 302 conductively coupled to the PCB 208 and the acoustic stack formed thereon, the transducer assembly 300 may be folded so as to vertically stack the PCB 208 and the piezoelectric crystal body 302. For example, a first fold may be made along the side 210 of the PCB 208 to orient the flex circuit 202 vertically. As used herein, when an object, feature, or component is oriented it is approximately parallel with the direction of an axis or a plane. For example, when the flex circuit 202 is oriented vertically, it is approximately parallel with the y-axis. The first fold may be a downward fold with reference to the axis system 299. A second fold may be made along a line 304 to orient the flex circuit 202 and piezoelectric crystal body 302 mounted thereon horizontally in an x-z plane with reference to the axis system 299. In this way, the transducer assembly 300 provides a compact package.

FIG. 4 shows an ultrasound probe 400. The ultrasound probe 400 comprises a housing 402, the transducer assembly 300 positioned inside the housing, and a battery 416 coupled to the transducer assembly 300. Components of the transducer assembly 300 introduced with reference to FIG. 3 are numbered the same and will not be reintroduced. Likewise, components of the flexible printed circuit board 200 introduced with reference to FIG. 2 are numbered the same and will not be reintroduced. The ultrasound probe 400 may be communicatively coupled to subsystems of a medical imaging system via one or more wireless networks, including, for example, a user interface, a medical records system, and a remote connectivity subsystem. In some examples, the ultrasound probe 400 may be the same or similar to the ultrasound probe 106 described with reference to the medical imaging system 100 in FIG. 1.

In one example, the housing 402 approximately conforms to a shape of the transducer assembly 300 positioned therein. The housing 402 may be a layered construction including, for example, a plastic case exterior, a metal shield, and an acoustic insulator. In one example, the housing 402 may comprise a phase change chamber configured to thermally interface with one or more heat generating components in the ultrasound probe 400 to dissipate the heat generated by the components of the ultrasound probe 400. For example, the housing 402 may include material configured to change phase, e.g., a solid phase and a liquid phase, in response to heat received from a component of the ultrasound probe 400. In some examples, the ultrasound probe 400 may be controlled to cycle between scanning, e.g., for a few seconds, and stopping scanning, e.g., for a few minutes. Inclusion of phase change material in the housing 402 may distribute the heat dissipation over time. The ultrasound probe 400 may be attached or coupled to a patient variously. For example, the ultrasound probe 400 may be affixed directly to a patient using one or more of a bandage, a strap, and an adhesive. Adhesive 436 applied to a peripheral portion 418 is shown schematically.

The ultrasound probe 400 includes a piezoelectric transducer 410. In one example, the piezoelectric transducer 410 may comprise the piezoelectric crystal body 302 formed on the top electrode 212 of the flex circuit 202. Further, the piezoelectric transducer 410 may comprise one or more additional layers to form a transducer structure. For example, the transducer structure or acoustic stack may include an acoustic backing material, a top electrode, a PZT crystal body, a bottom electrode, one or more matching layers, and/or a lens aligned along the y-axis. In one example, the lens may be positioned at the peripheral portion 418 of the transducer housing 402 through which ultrasound radiation may be transmitted and received. The acoustic stack is acoustically coupled to the lens, positioned along the y-axis so as to not obstruct receipt of ultrasonic waves by the lens. The acoustic stack may include an array of piezoelectric elements, such as, for example, the piezoelectric crystal body 302, for converting received ultrasound vibrations into voltage signals and for converting received voltage signals into ultrasound vibrations. The acoustic backing material may be bonded to a side of the acoustic stack facing away from the lens. In one example, the transducer structure may include a bottom electrode (called a ground electrode) applied to the piezoelectric crystal body 302 that may be electrically coupled to the flex circuit 202 to via a ground return connection to complete the circuit. The coupling of the ground return to the flex circuit 202, may be achieved through direct connection during assembly. For example, the ultrasound probe 400 may include a jumper wire coupling the flex layer and the bottom electrode applied to the piezoelectric crystal body 302.

The ultrasound probe 400 includes the PCB 208. In one example, the PCB 208 includes a core PCB 408, a top shielding layer 414, and a bottom shielding layer 412. An insulating layer 404 is arranged above the PCB 208. The insulating layer 404 may be above and in face sharing contact with the top shielding layer 414. The insulating layer 404 may be sandwiched between the top shielding layer 414 and an interior surface of the housing 402. The flex circuit 202 is integral with the core PCB 408. In one example, the flex circuit 202 is folded into a vertical plane 424 and a horizontal plane 426. The horizontal plane 426 comprises the top electrode 212 on which the piezoelectric transducer 410 is built. Folded in this way, a surface 430, such as a lens, of the piezoelectric transducer 410 faces downward in the direction of the peripheral portion 418 of the ultrasound probe 400. In one example, the flex circuit 202 further includes a flex circuit tab 428 to facilitate case of connection to the battery 416.

The ultrasound probe 400 includes the battery 416 electrically coupled to the transducer assembly 300. In one example, the battery 416 is coupled to the PCB 208 via the flex circuit tab 428. The battery 416 is interposed between the piezoelectric transducer 410 and the PCB 208. For example, the battery 416 is arranged below the bottom shielding layer 412, e.g., with nothing in between, and above piezoelectric transducer 410 with the flex circuit 202 therebetween. In one example, the ultrasound probe 400 may include thermistors to measure heating and limit operation in response to detecting heat greater than a threshold.

Referring now to FIG. 5, a second example of a flexible printed circuit board 500 that may be included in a transducer assembly is schematically depicted. In one example, the flexible printed circuit board 500 may be the same or similar to the PCB 150 included in the ultrasound probe 106 shown in FIG. 1.

The flexible printed circuit board 500 is configured similarly to the flexible printed circuit board 200. For example, the flexible printed circuit board 500 may include a flex circuit 502 that is integral with a main circuit board assembly (PCB) 508. The flex circuit 502 includes an electrode pattern 506 directly integrated into the flex material, e.g., a polymide or suitable material. The electrode pattern 506 may include a top electrode 512 and a ground return connection 514 directly integrated into the flex circuit 502. The electrode pattern 506 may be conductively coupled to the PCB 508 via a plurality of electrical traces 504.

The flex circuit 502 extends laterally from a side 510 of the PCB 508. A piezoelectric crystal body, such as piezoelectric crystal body 302, may be directly attached to the top electrode 512 and the transducer formed thereon, e.g., additional layers applied thereto, if used. For example, a bottom electrode (or ground electrode) may be applied to the piezoelectric crystal body 302. The flex circuit 502 may be folded along a line 518 to arrange the ground return connection 514 in face sharing contact with the transducer, e.g., with the bottom electrode of the transducer. The flex circuit 502 may include additional folds to vertically stack the PCB 508 and the transducer. For example, a first fold, which may be a downward fold with reference to the axis system 299, may be made along the side 510 of the PCB 508 to orient the flex circuit 502 vertically. A second fold may be made along a line 516 to orient the flex circuit 502 and the transducer mounted thereon horizontally in an x-z plane with reference to the axis system 299.

In one example, the PCB 508 may comprise the main circuit board of the ultrasound control system described with reference to FIG. 1. For example, the PCB 508 may include one or more of transmitter and receiver channels for generating and processing ultrasound signals, analog-to-digital converters for converting the analog transducer signals into digital signals, digital circuitry for performing beamforming, filtering, and other algorithms for processing the ultrasound data, memory including RAM and/or flash memory, Complex Programmable Logic Devices (CPLD), and control circuity including one or more microcontrollers. The PCB 208 may be prepared with custom ASICs to integrate ultrasound control functions, increase performance, and increase efficiency.

FIG. 6 shows an ultrasound probe 600. The ultrasound probe 600 comprises a housing 602, a transducer assembly 606 positioned inside the housing, and a battery 616 coupled to the transducer assembly 606. The ultrasound probe 600 may be communicatively coupled to subsystems of a medical imaging system via one or more wireless networks, including, for example, a user interface, a medical records system, and a remote connectivity subsystem. In some examples, the ultrasound probe 600 may be the same or similar to the ultrasound probe 106 described with reference to the medical imaging system 100 in FIG. 1. The components included in the ultrasound probe 600 may be similar to the components described with reference to the ultrasound probe 400 in FIG. 4.

In one example, the housing 602 approximately conform to a shape of the transducer assembly 606 positioned therein. The housing 602 may be a layered construction including, for example, a plastic case exterior, a metal shield, an acoustic insulator, and in some examples, may further include a phase change chamber configured to dissipate the heat generated by the components of the ultrasound probe 600, such as described with reference to FIG. 4. In some examples, the ultrasound probe 600 may be bandaged, wrapped, or glued to a patient, such as described with reference to FIG. 4. Adhesive 636 applied to a peripheral portion 618 is shown schematically.

The transducer assembly 606 may be similar to the transducer assembly 300. For example, the transducer assembly 606 comprises the flex circuit 502 and the PCB 508, the electrode pattern 506 directly integrated into the flex circuit 502, and a piezoelectric transducer 610 directly attached to the electrode pattern 506. In one example, the piezoelectric transducer 610 may comprise the piezoelectric crystal body 302 of FIG. 3 formed on the top electrode 512 of the flex circuit 502. Further, the transducer assembly 606 may comprise one or more additional layers to form a transducer structure, such as, for example, acoustic backing material, one or more matching layers, and/or a lens aligned along the y-axis. The transducer structure may include a bottom electrode applied to the piezoelectric crystal body 302 that may be electrically coupled to the PCB 508 via a ground return connection 514. In contrast to ultrasound probe 400, the electrical coupling between the bottom electrode patterned on the piezoelectric crystal body and the ground return connection 514 is made through folding and bonding the flex layer rather than with a jumper wire.

In one example, the PCB 508 includes a core PCB 608, a top shielding layer 614, and a bottom shielding layer 612. An insulating layer 604 is arranged above the PCB 508. For example, the insulating layer 604 may be sandwiched between the top shielding layer 614 and an interior surface of the housing 602. The flex circuit 502 is integral with the core PCB 608. In one example, the flex circuit 202 further includes a flex circuit tab 632 to provide the ground return connection. In one example, the flex circuit 502 is folded into a first vertical plane 624, a first horizontal plane 626, a second vertical plane 628, and a second horizontal plane 630. The first horizontal plane 626 comprises the top electrode 512 on which the piezoelectric transducer 610 is built. The second horizontal plane 630 comprises the ground return connection 514 in face sharing contact with the piezoelectric transducer 610. Folded in this way, a surface 634, such as a lens, of the piezoelectric transducer 610 faces downward in the direction of the peripheral portion 618 of the ultrasound probe 600.

The ultrasound probe 600 includes the battery 616 electrically coupled to the transducer assembly 606. In one example, the battery 616 is coupled to the PCB 508 via the flex circuit tab 632. The battery 616 is interposed between the piezoelectric transducer 610 and the PCB 508. For example, the battery 616 is arranged below the bottom shielding layer 612, e.g., with nothing in between, and above piezoelectric transducer 610 with the flex circuit 502 therebetween.

In this way, the ultrasound probe 600 provides a durable and compact package, including a top electrode and ground return connection, without using a detachable connector, cables, or other standard conductive couplings between the main circuit assembly and the transducer.

Referring now to FIG. 7, a third example of a flexible printed circuit board 700 that may be included in a transducer assembly is schematically depicted. In one example, the flexible printed circuit board 700 may be the same or similar to the PCB 150 included in the ultrasound probe 106 shown in FIG. 1.

The flexible printed circuit board 700 is configured similarly to the flexible printed circuit board 200 and the flexible printed circuit board 500 described with reference to FIG. 2 and FIG. 5, respectively. For example, the flexible printed circuit board 700 may include a flex circuit 702 that is integral with a main circuit board assembly (PCB) 708. The flex circuit 702 includes an electrode pattern 706 directly integrated into the flex material, e.g., a polymide or suitable material. The electrode pattern 706 may include a first electrode and a second electrode of the transducer assembly directly integrated into the flex circuit 702. For example, the first electrode may be a top electrode 712 and the second electrode may be a bottom electrode 714. The electrode pattern 706 may be conductively coupled to the PCB 708 via a plurality of electrical traces 704.

The flex circuit 702 extends laterally from a side 710 of the PCB 708. A piezoelectric crystal body, such as piezoelectric crystal body 302 of FIG. 3, may be directly attached to the top electrode 712 and the transducer formed thereon, e.g., additional layers applied thereto, if used. The flex circuit 202 may be folded along a line 718 and a line 720 to arrange the bottom electrode 714 in face sharing contact with the transducer. The flex circuit 702 may include additional folds to vertically stack the PCB 708 and the transducer. For example, a third fold, which may be a downward fold with reference to the axis system 299, may be made along the side 710 of the PCB 708 to orient the flex circuit 702 vertically. A fourth fold may be made along a line 716 to orient the flex circuit 702 and the transducer mounted thereon horizontally in an x-z plane with reference to the axis system 299.

In one example, the PCB 708 may comprise the main circuit board of the ultrasound control system described with reference to FIG. 1. For example, the PCB 708 may include one or more of transmitter and receiver channels for generating and processing ultrasound signals, analog-to-digital converters for converting the analog transducer signals into digital signals, digital circuitry for performing beamforming, filtering, and other algorithms for processing the ultrasound data, memory including RAM and/or flash memory, Complex Programmable Logic Devices (CPLD), and control circuity including one or more microcontrollers. The PCB 208 may be prepared with custom ASICs to integrate ultrasound control functions, increase performance, and increase efficiency.

FIG. 8 shows an ultrasound probe 800. The ultrasound probe 800 comprises a housing 802, a transducer assembly 806 positioned inside the housing, and a battery 816 coupled to the transducer assembly 806. The ultrasound probe 800 may be communicatively coupled to subsystems of a medical imaging system via one or more wireless networks, including, for example, a user interface, a medical records system, and a remote connectivity subsystem. In some examples, the ultrasound probe 800 may be the same or similar to the ultrasound probe 106 described with reference to the medical imaging system 100 in FIG. 1. The components included in ultrasound probe 800 may be similar to the components described with reference to ultrasound probe 400 in FIG. 4 and the ultrasound probe 600 in FIG. 6.

In one example, the housing 802 approximately conforms to a shape of the transducer assembly 806 positioned therein. The housing 802 may be a layered construction including, for example, a plastic case exterior, a metal shield, and an acoustic insulator, and in some examples, may include a phase change chamber configured to dissipate the heat generated by the components of the ultrasound probe 800. In some examples, the ultrasound probe 800 may be bandaged, wrapped, or glued to a patient, such as described with reference to FIG. 4 and FIG. 6. Adhesive 836 applied to a peripheral portion 818 is shown schematically.

The transducer assembly 806 may be similar to the transducer assembly 300. For example, the transducer assembly 806 includes the flexible printed circuit board 700 comprising the flex circuit 702 and the PCB 708, the electrode pattern 706 directly integrated into the flex circuit 702, and a piezoelectric transducer 810 directly attached to the electrode pattern 706. In one example, the piezoelectric transducer 810 may comprise the piezoelectric crystal body 302 formed on the top electrode 712 and the bottom electrode 714 attached to an opposing side of the piezoelectric crystal body 302. In some examples, the piezoelectric transducer 810 may comprise one or more additional layers to form a transducer structure, such as, for example, acoustic backing material, an acoustic stack, one or more matching layers, and/or a lens aligned along the y-axis. In contrast to the ultrasound probe 400 and the ultrasound probe 600, the bottom electrode 714 coupling between the PCB 708 and the piezoelectric crystal body is entirely formed by wrapping the piezoelectric crystal body in the flex layer.

In one example, the PCB 708 includes a core PCB 808, a top shielding layer 814, and a bottom shielding layer 812. An insulating layer 804 is arranged above the core PCB 808. For example, the insulating layer 804 may be sandwiched between the top shielding layer 814 and an interior surface of the housing 802. The flex circuit 702 is integral with the core PCB 808. In one example, the flex circuit 202 further includes a flex circuit tab 832. In one example, the flex circuit 702 is folded into a first vertical plane 824, a first horizontal plane 826, a second vertical plane 828, and a second horizontal plane 830. The first horizontal plane 826 comprises the top electrode 712 on which the piezoelectric transducer 810 is built. The second horizontal plane 830 comprises the bottom electrode 714, replacing any additional processing steps to pattern the piezoelectric transducer 810 with a ground return contact. Folded in this way, a surface 834, such as a lens, of the piezoelectric transducer 810 faces downward in the direction of the peripheral portion 818 of the ultrasound probe 800.

The ultrasound probe 800 includes the battery 816 electrically coupled to the transducer assembly 806. In one example, the battery 816 is coupled to the core PCB 808 via the flex circuit tab 832. The battery 816 is interposed between the piezoelectric transducer 810 and the core PCB 808. For example, the battery 816 is arranged below the bottom shielding layer 812, e.g., with nothing in between, and above piezoelectric transducer 810 with the flex circuit 702 therebetween.

In this way, the ultrasound probe 800 provides a durable and compact package, including a top electrode and bottom electrode, without using a detachable connector, cables, or other standard conductive couplings between the main circuit assembly and the transducer.

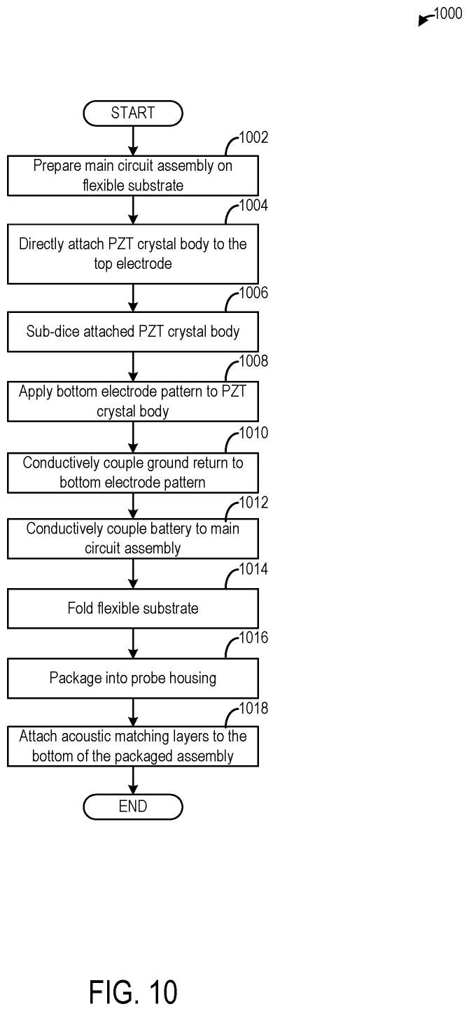

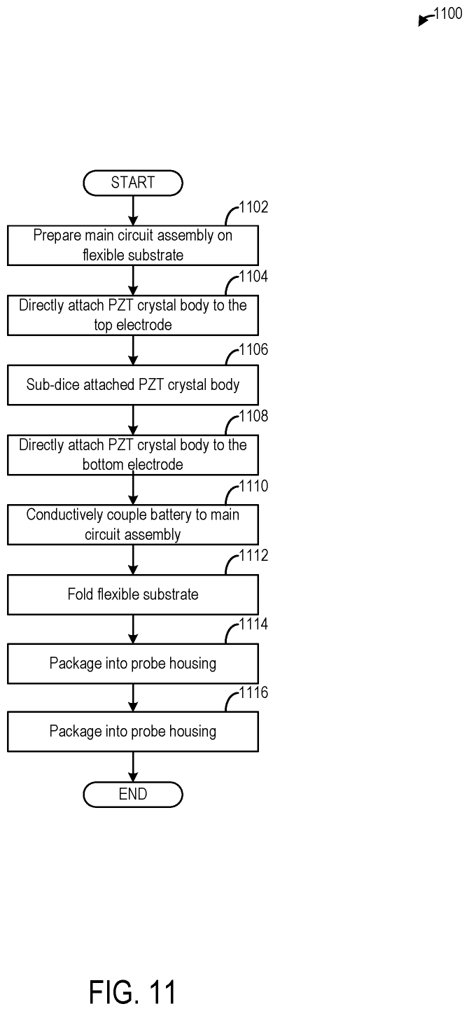

FIG. 9, FIG. 10, and FIG. 11 are flow charts of methods 900, 1000, and 1100, respectively, for manufacturing an ultrasound transducer assembly including forming a piezoelectric transducer directly on a flex circuit of a main circuit assembly without anything in between. The method 900 is described below with regard to the examples depicted in FIGS. 2-4, the method 1000 with regard to the examples depicted in FIGS. 5-6, and the method 1100 with regard to the examples depicted in FIGS. 7-8; however, it should be appreciated that methods 900, 1000, and 1100 may be implemented to manufacture other examples without departing from the scope of the present disclosure.

Referring now to FIG. 9, the method 900 may be employed to manufacture the ultrasound probe 400 of FIG. 4.

At 902, the method 900 may include preparing a main circuit board assembly on a flexible or mixed rigid-flex substrate. In one example, the flex portion of the assembly may comprise an elongate flexible segment having a plurality of electrical traces routed therethrough, the plurality of electrical traces conductively coupled to a top electrode pattern integrated therein. In this step standard electronics assembly also occurs.

At 904, the method 900 may include directly attaching a lead-zircon-titanate (PZT) crystal body, or other suitable piezoelectric material, to the top electrode pattern. For example, direct attachment may include fixing the PZT crystal body to the top electrode pattern using one of thermocompression, ACP, ACF, and non-conductive epoxy.

At 906, the method 900 may include sub-dicing of the attached piezoelectric crystal body.

At 908, the method 900 may include applying a bottom electrode pattern to the PZT crystal body. For example, a conductive layer such as may be formed on a front face of the crystal by plating a thin film of gold or silver on the surfaces.

At 910, the method 900 may include conductively coupling the bottom electrode to the main circuit board assembly for example via a wire bond connection.

At 912, the method 900 may include conductively coupling a battery to the main circuit board assembly. For example, the battery may be coupled via a length of the flex circuit.

At 914, the method 900 may include folding the flexible substrate. For example, the flexible substrate may be folded so as to vertically stack the transducer and the main circuit board assembly.

At 916, the method 900 may include packing the transducer assembly and battery coupled thereto into a probe housing. For example, the housing may be a layered construction including, for example, a plastic case exterior, a metal shield, an acoustic insulator, and in some examples, may include a phase change chamber.

At 918, the method 900 may include attachment of acoustic matching layer materials on to the bottom of the packaged assembly.

Referring now to FIG. 10, the method 1000 may be employed to manufacture the ultrasound probe 600 of FIG. 6.

At 1002, the method 1000 may include preparing a main circuit board assembly on a on a flexible or mixed rigid-flex substrate. In one example, the flex portion of the assembly may comprise an elongate flexible segment having a plurality of electrical traces routed therethrough, the plurality of electrical traces conductively coupled to a top electrode pattern and a ground return connection integrated therein. In this step standard electronics assembly also occurs.

At 1004, the method 1000 may include directly attaching a lead-zircon-titanate (PZT) crystal body, or other suitable piezoelectric material, to the top electrode. For example, direct attachment may include fixing the PZT crystal body to the top electrode pattern using one of thermocompression, ACP, ACF, and non-conductive epoxy.

At 1006, the method 1000 may include sub-dicing of the attached piezoelectric crystal body.

At 1008, the method 1000 may include applying a bottom electrode pattern to the PZT crystal body. For example, a conductive layer such as may be formed on a front face of the crystal by plating a thin film of gold or silver on the surfaces.

At 1010, the method 1000 may include conductively coupling the ground return connection to the bottom electrode to the main circuit board assembly. Similarly, the ground return connection may be coupled to the bottom electrode using standard attachment approaches such as thermocompression, etc.

At 1012, the method 1000 may include conductively coupling a battery to the main circuit board assembly. For example, the battery may be coupled to a length of the flex circuit.

At 1014, the method 1000 may include folding the flexible substrate. For example, the flexible substrate may be folded so as to vertically stack the transducer and the main circuit board assembly.

At 1016, the method 1000 may include packing the transducer assembly and battery coupled thereto into a probe housing. For example, the housing may be a layered construction including, for example, a plastic case exterior, a metal shield, an acoustic insulator, and in some examples, may include a phase change chamber.

At 1018, the method 1000 may include attachment of acoustic matching layer materials on to the bottom of the packaged assembly.

Referring now to FIG. 11, the method 1100 may be employed to manufacture the ultrasound probe 800 of FIG. 8.

At 1102, the method 1100 may include preparing a main circuit board assembly on a flexible or mixed rigid-flex substrate. In one example, the flex portion of the assembly may comprise an elongate flexible segment having a plurality of electrical traces routed therethrough, the plurality of electrical traces conductively coupled to a top electrode pattern and a bottom electrode integrated therein. In this step standard electronics assembly also occurs.

At 1104, the method 1100 may include directly attaching a lead-zircon-titanate (PZT) crystal body, or other suitable piezoelectric material, to the top electrode. For example, direct attachment may include fixing the PZT crystal body to the top electrode pattern using one of thermocompression, ACP, ACF, and non-conductive epoxy. The top electrode may couple to a rear face of the PZT crystal body.

At 1106, the method 1100 may include sub-dicing of the attached piezoelectric crystal body.

At 1108, the method 1100 may include directly attaching the PZT crystal body to the bottom electrode. Similarly, the PZT crystal body may be coupled to the bottom electrode using standard attachment approaches such as thermocompression, etc. The bottom electrode may couple to a front face of the PZT crystal body.

At 1110, the method 1100 may include conductively coupling a battery to the main circuit board assembly. For example, the battery may be coupled to a length of the flex circuit.

At 1112, the method 1100 may include folding the flexible substrate. For example, the flexible substrate may be folded so as to vertically stack the transducer and the main circuit board assembly.

At 1114, the method 1100 may include packing the transducer assembly and battery coupled thereto into a probe housing. For example, the housing may be a layered construction including, for example, a plastic case exterior, a metal shield, an acoustic insulator, and in some examples, may include a phase change chamber.

At 1116, the method 1000 may include attachment of acoustic matching layer materials on to the bottom of the packaged assembly.

FIG. 12 shows a fourth example of a flexible printed circuit board 1200 that may be included in a transducer assembly. The flexible printed circuit board 1200 may be the same or similar to the PCB 150 included in the ultrasound probe 106 shown in FIG. 1.

The flexible printed circuit board 1200 is configured similarly to the flexible printed circuit board 200, the flexible printed circuit board 500, and the flexible printed circuit board 700 described with reference to FIG. 2, FIG. 5, and FIG. 7, respectively. For example, the flexible printed circuit board 1200 may include a flex circuit 1202 that is integral with a main circuit board assembly (PCB) 1208. The flex circuit 1202 includes an electrode pattern 1206 directly integrated into the flex material, e.g., a polymide or suitable material. The electrode pattern 1206 may include a top electrode 1212 and a bottom electrode 1214 directly integrated into the flex circuit 1202. The electrode pattern 1206 may be conductively coupled to the PCB 1208 via a plurality of electrical traces 1204. In contrast with the flexible printed circuit board 700, the top electrode 1212 may comprise a ground return and the bottom electrode 1214 may comprise a plurality of clement traces. Configured in this manner, the ground return is arranged closer to the PCB 1208 and the element traces are arranged further from the PCB 1208. Such a configuration may simplify manufacturing in some examples.

In this way, a piezoelectric transducer is built directly on a main circuit board assembly of an ultrasound imaging system thereby avoiding use of bulky, expensive, and fragile detachable connectors. The technical effect is an ultra-compact ultrasound probe with increased durability.

The disclosure also provides support for a method, comprising: forming a transducer directly on a flex circuit of a main circuit assembly without anything in between, wherein the transducer is a piezoelectric transducer. In a first example of the method, the method further comprises: directly integrating an electrode pattern into the flex circuit. In a second example of the method, optionally including the first example, the forming comprises attaching a piezoelectric crystal body directly to the electrode pattern using one of thermocompression, ACF, ACP, and non-conductive epoxy. In a third example of the method, optionally including one or both of the first and second examples, the flex circuit comprises an elongate flexible segment having a plurality of electrical traces routed therethrough, the plurality of electrical traces conductively coupled to the electrode pattern integrated therein. In a fourth example of the method, optionally including one or more or each of the first through third examples, the method further comprises: folding the flex circuit so as to vertically stack the transducer and the main circuit assembly. In a fifth example of the method, optionally including one or more or each of the first through fourth examples, the electrode pattern is an electrode of the transducer. In a sixth example of the method, optionally including one or more or each of the first through fifth examples, the electrode pattern comprises an electrode on two sides of the transducer. In a seventh example of the method, optionally including one or more or each of the first through sixth examples, the electrode pattern is an electrode and a ground return connection. In an eighth example of the method, optionally including one or more or each of the first through seventh examples, the method further comprises: sub-dicing the attached piezoelectric crystal body.

The disclosure also provides support for an ultrasound probe, comprising: a housing, a transducer assembly positioned inside the housing, the transducer assembly comprising: a flexible printed circuit board comprising a flex circuit and a main circuit board assembly, an electrode pattern directly integrated into the flex circuit, and a piezoelectric transducer directly attached to the electrode pattern, and, a battery electrically coupled to the transducer assembly. In a first example of the system, the housing is a layered construction comprising one or more of a plastic case exterior, a metal shield, an acoustic insulator, and phase change chamber. In a second example of the system, optionally including the first example, the electrode pattern further comprises one of a bottom electrode and a ground return connection of the transducer assembly. In a third example of the system, optionally including one or both of the first and second examples, the piezoelectric transducer is conductively coupled to the main circuit board assembly via the flex circuit without a detachable connector. In a fourth example of the system, optionally including one or more or each of the first through third examples, the ultrasound probe is communicatively coupled to a user interface via one or more wireless networks. In a fifth example of the system, optionally including one or more or each of the first through fourth examples, the ultrasound probe is patch probe affixed directly to a patient using one or more of a bandage, a strap, and an adhesive.

The disclosure also provides support for an imaging system, comprising: an ultrasound probe, comprising: a housing, a transducer assembly positioned inside the housing, the transducer assembly comprising: a flexible printed circuit board comprising a flex circuit and a main circuit board assembly, an electrode pattern directly integrated into the flex circuit, and a piezoelectric transducer directly attached to the electrode pattern, and, a battery electrically coupled to the transducer assembly, and, a user interface communicatively coupled with the ultrasound probe via one or more wireless networks, the user interface comprising a display device and a user input device, wherein the main circuit board assembly is configured to acquire image data corresponding to a subject and to process the image data to generate one or more datasets corresponding to the subject. In a first example of the system, the housing is a layered construction comprising one or more of a plastic case exterior, a metal shield, an acoustic insulator, and phase change chamber. In a second example of the system, optionally including the first example, the electrode pattern is a first electrode of the transducer assembly. In a third example of the system, optionally including one or both of the first and second examples, the electrode pattern further comprises one of a second electrode and a ground return connection of the transducer assembly. In a fourth example of the system, optionally including one or more or each of the first through third examples, the piezoelectric transducer comprises a piezoelectric crystal body conductively coupled to the main circuit board assembly via the flex circuit without a detachable connector.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects. As used herein, the term “approximately” is construed to mean plus or minus five percent of the range unless otherwise specified.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. A method, comprising:
forming a transducer directly on a flex circuit of a main circuit assembly without anything in between, wherein the transducer is a piezoelectric transducer.

2. The method of claim 1, further comprising, directly integrating an electrode pattern into the flex circuit.

3. The method of claim 2, wherein the forming comprises attaching a piezoelectric crystal body directly to the electrode pattern using one of thermocompression, ACF, ACP, and non-conductive epoxy.

4. The method of claim 2, wherein the flex circuit comprises an elongate flexible segment having a plurality of electrical traces routed therethrough, the plurality of electrical traces conductively coupled to the electrode pattern integrated therein.

5. The method of claim 1, further comprising, folding the flex circuit so as to vertically stack the transducer and the main circuit assembly.

6. The method of claim 2, wherein the electrode pattern is an electrode of the transducer.

7. The method of claim 2, wherein the electrode pattern comprises an electrode on two sides of the transducer.

8. The method of claim 2, wherein the electrode pattern is an electrode and a ground return connection.

9. The method of claim 3, further comprising sub-dicing the attached piezoelectric crystal body.

10. An ultrasound probe, comprising:
a housing;
a transducer assembly positioned inside the housing, the transducer assembly comprising:
a flexible printed circuit board comprising a flex circuit and a main circuit board assembly;
an electrode pattern directly integrated into the flex circuit; and
a piezoelectric transducer directly attached to the electrode pattern; and,
a battery electrically coupled to the transducer assembly.

11. The ultrasound probe of claim 10, wherein the housing is a layered construction comprising one or more of a plastic case exterior, a metal shield, an acoustic insulator, and phase change chamber.

12. The ultrasound probe of claim 10, wherein the electrode pattern further comprises one of a bottom electrode and a ground return connection of the transducer assembly.

13. The ultrasound probe of claim 10, wherein the piezoelectric transducer is conductively coupled to the main circuit board assembly via the flex circuit without a detachable connector.

14. The ultrasound probe of claim 10, wherein the ultrasound probe is communicatively coupled to a user interface via one or more wireless networks.

15. The ultrasound probe of claim 10, wherein the ultrasound probe is patch probe affixed directly to a patient using one or more of a bandage, a strap, and an adhesive.

16. An imaging system, comprising:
an ultrasound probe, comprising:
a housing;
a transducer assembly positioned inside the housing, the transducer assembly comprising:
a flexible printed circuit board comprising a flex circuit and a main circuit board assembly;
an electrode pattern directly integrated into the flex circuit; and
a piezoelectric transducer directly attached to the electrode pattern; and,
a battery electrically coupled to the transducer assembly; and,
a user interface communicatively coupled with the ultrasound probe via one or more wireless networks, the user interface comprising a display device and a user input device,
wherein the main circuit board assembly is configured to acquire image data corresponding to a subject and to process the image data to generate one or more datasets corresponding to the subject.

17. The imaging system of claim 16, wherein the housing is a layered construction comprising one or more of a plastic case exterior, a metal shield, an acoustic insulator, and phase change chamber.

18. The imaging system of claim 16, wherein the electrode pattern is a first electrode of the transducer assembly.

19. The imaging system of claim 18, wherein the electrode pattern further comprises one of a second electrode and a ground return connection of the transducer assembly.

20. The imaging system of claim 16, wherein the piezoelectric transducer comprises a piezoelectric crystal body conductively coupled to the main circuit board assembly via the flex circuit without a detachable connector.
